(19) United States
(12) Patent Application Publication
(10) Pub. No.: US 2022/0395393 A1
(43) Pub. Date: Dec. 15, 2022

(54) HANDPIECE INCLUDING COOLING DEVICE

(71) Applicant: Sun-Young KANG, Seoul (KR)

(72) Inventor: Sun-Young KANG, Seoul (KR)

(21) Appl. No.: 17/684,449

(22) Filed: Mar. 2, 2022

(30) Foreign Application Priority Data
Jun. 9, 2021 (KR) 10-2021-0074491

(51) Int. Cl. A61F 7/00; A45D 44/00

(52) U.S. Cl. CPC A61F 7/007; A45D 44/00; A61F 2007/0052; A61F 2007/0056

(57) ABSTRACT

A handpiece including a cooling device for a skin care device used for improving or treating a skin. The handpiece includes: a case; a cooling head part mounted at a fore-end inside the case and configured to generate cooling energy; a cooling plate part which has a rear end for receiving the cooling energy from the cooling head part and has a fore-end that cools a treatment site through the contact with the skin; a needle part which includes needles for penetrating into the skin during the procedure for skin care; and a linear motor unit which includes a driving unit configured to penetrate the cooling head part is coupled to the needle, and a motor unit located at a rear of the cooling head part and inducing a linear movement of the needle part by a forward and backward movement of a driving unit.

### CROSS-REFERENCE TO RELATED APPLICATIONS

This application claims priority to and the benefit of Korean Patent Application No. 10-2021-0074491 filed in the Korean Intellectual Property Office on Jun. 9, 2021, the entire contents of which are incorporated herein by reference.

### TECHNICAL FIELD

The present invention relates to a handpiece including a cooling device, and more particularly, to a handpiece including a cooling device which cools the skin, relieves pain, and has a soothing effect on the skin, and improves cooling performance and efficiency through improvement of heat transfer mechanism.

### BACKGROUND ART

Recently, interest in skin beauty and treatment is increasing, and various devices for the skin care are being developed and release.

Skin care refers to improving and treating the skin in such a way as to modify or eliminate the condition of the skin tissue by transmitting or applying various types of stimuli to the skin side, and uses various forms of electromagnetic waves, including laser beams, flash lamps, RF high frequency, microwaves, and ultrasound.

Herein, the RF high frequency method is the method of improving and treating the skin by generating frictional heat by high frequency in the tissue of the skin by applying high frequency to the skin through needles in the state where the needles are penetrated into the skin, and changing the condition of the tissue by applying local thermal damage by the thermal energy of the frictional heat.

That is, depending on the wavelength characteristics of electromagnetic waves, skin improvement and treatment effects may be different according to the procedure.

As described above, in the improvement and treatment of the skin by using the skin care devices, the thermal energy generated by transmitting or applying various types of stimuli may cause damage, including burns, to the skin surface. Therefore, the technology for cooling the skin during the procedure of the skin care is applied together.

In the meantime, in the related art, as the skin cooling technology grafted into the skin care device, a method of cooling the skin by including a cooling device and contacting the cooling device to the skin is mainly used.

However, in the cooling device for the skin care device in the related art, it may be difficult to integrally manufacture the cooling device according to a structure of a handpiece, and even though the cooling device is integrally provided onto the handpiece, there is a problem in that the cooling device occupies a large space.

In particular, in the cooling method grafted to the handpiece in the related art, there is a problem in that the cooling effect that is the most important point deteriorates or is inevitably extremely limited.

### PRIOR ART LITERATURE

### [Patent Document]

(Patent Document 1) Korean Patent No. 10-1232708

(Patent Document 2) Korean Patent No. 10-1117198

### SUMMARY OF THE INVENTION

The present invention is conceived in response to the background art, and has been made in an effort to provide a handpiece including a cooling device, which cools a skin to relieve pain and exhibit a skin soothing effect.

The present invention is also conceived in response to the background art, and has been made in an effort to provide a handpiece for a skin care device, which includes a cooling device integrated inside the handpiece, and improves cooling performance and efficiency by minimizing heat loss through improvement of heat transfer mechanism.

The present invention is also conceived in response to the background art, and has been made in an effort to provide a handpiece including a cooling device, in which a driving unit for forward and backward driving of needles is implemented to penetrate a cooling device to design optimized and compact space.

An exemplary embodiment of the present invention provides a handpiece including a cooling device for a skin care device used for improving or treating a skin, the handpiece including: a case provided for an appearance of the handpiece; a cooling head part mounted at a fore-end inside the case and configured to perform a heat exchange function and generate cooling energy; a cooling plate part which has a rear end that is in contact with the cooling head part to receive the heat-exchanged cooling energy from the cooling head part and has a fore-end that is positioned in front of the case and is exposed to cool a treatment site through the contact with the skin during a procedure for skin care; a needle part which is located at a fore-end of the cooling head part or located in front of the cooling head part, and includes needles for penetrating into the skin during the procedure for skin care; and a linear motor unit which includes a driving unit disposed so as to penetrate the cooling head part is coupled to the needle, and a motor unit located at a rear of the cooling head part, and induces a forward and backward linear movement of the driving unit.

A coolant movement line may be connected to a rear end of the case to circulate and supply the coolant, and the coolant may be introduced to the cooling head part and be used as a medium for performing a heat exchange function.

The cooling head part may include: a heat exchange block which is located at a fore-end inside the case, has a through-type body, and is formed with a coolant line for inflow and circulation of a coolant; one pair of Peltier elements disposed in close contact with upper and lower surfaces of the heat exchange block, respectively; one pair of cooling plate sockets which are disposed in the Peltier elements in a stack form, respectively, are fastened and fixed to the heat exchange block, and have plate insertion grooves to which a rear end of the cooling plate part is inserted and placed while being in contact with the cooling plate socket; and one pair of plate-type elastic bodies seated on upper surfaces of the cooling plate sockets, respectively, and interposed between the case and the cooling plate socket.

The cooling plate part may include: one pair of cooling energy transfer plates of which rear ends are inserted into the cooling plate sockets of the cooling head part to be in contact with the cooling plate sockets, respectively, and fore-ends are exposed to the outside of the case, and which are located at upper and lower sides and disposed at an interval; and a skin contact cooling plate which is coupled to the pore-ends of the one pair of cooling energy transfer plate exposed to the outside to function as connection members, and is in direct contact with the skin during procedure to serve to cool a treatment site, and is formed with needle through-holes for penetration and arrangement of the needles.

At least one detachment groove may be formed in an external surface of a rear end of each of the cooling energy transfer plates, detachment protrusions may be formed in each of the plate-type elastic bodies at positions corresponding to positions of the detachment grooves and in the number corresponding to the number of detachment grooves, and in the cooling plate part, when the cooling energy transfer plate is inserted into the cooling plate socket, the detachment protrusion of the plate-type elastic body may be inserted into the detachment groove of the cooling energy transfer plate to provide fixing force, and when the cooling energy transfer plate is pulled out from the cooling plate socket, a matched and fixed state of the detachment protrusion and the detachment groove may be easily released by inducing elasticity of the plate-type elastic body by a push operation by external pressure.

The plate-type elastic body may include a plate body seated on an upper surface of the cooling plate socket, and may be formed with one pair of slit holes disposed at an interval on the plate body for an elastic structure.

The heat exchange block may be made of an aluminum material, each of the Peltier elements may be disposed so that a heating surface is in close contact with the heat exchange block and a cooling surface is in close contact with the cooling plate socket, and the cooling plate socket may be made of a copper material so as to minimize thermal transfer loss for the cooling energy transferred to the cooling plate part.

According to the present invention, it is possible to provide the handpiece including the cooling device which cools the skin to relieve pain and exhibit a skin soothing effect, and in which the driving unit for forward and backward driving of the needles is implemented to penetrate the cooling device to achieve an optimized and compact space design.

According to the present invention, it is possible to provide the handpiece for a skin care device including the integrated cooling device therein, which is capable of preventing and minimizing heat loss through improvement of heat transfer mechanism, and thus improving cooling performance and efficiency.

### BRIEF DESCRIPTION OF THE DRAWINGS

FIG. 1 is a perspective view illustrating a handpiece including a cooling device according to an exemplary embodiment of the present invention.

FIG. 2 is a cross-sectional view illustrating the handpiece including the cooling device according to the exemplary embodiment of the present invention.

FIG. 3 is an exploded perspective view illustrating the handpiece including the cooling device according to the exemplary embodiment of the present invention.

FIG. 4 is a configurational diagram illustrating the main part representing the state where a case is removed in the handpiece including the cooling device according to the exemplary embodiment of the present invention.

FIG. 5 is a configuration diagram illustrating the state where a needle tip is removed in the drawing of FIG. 4.

### DETAILED DESCRIPTION

An exemplary embodiment of the present invention will be described with reference to the accompanying drawings, and an object and the configuration, and the features of the present invention will be understood well through the detailed description.

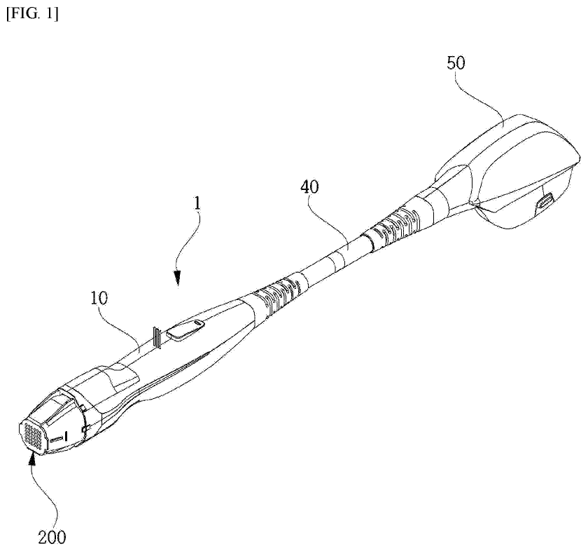

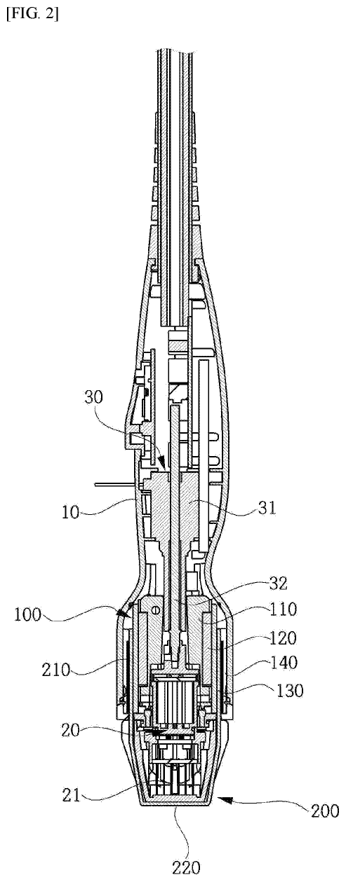

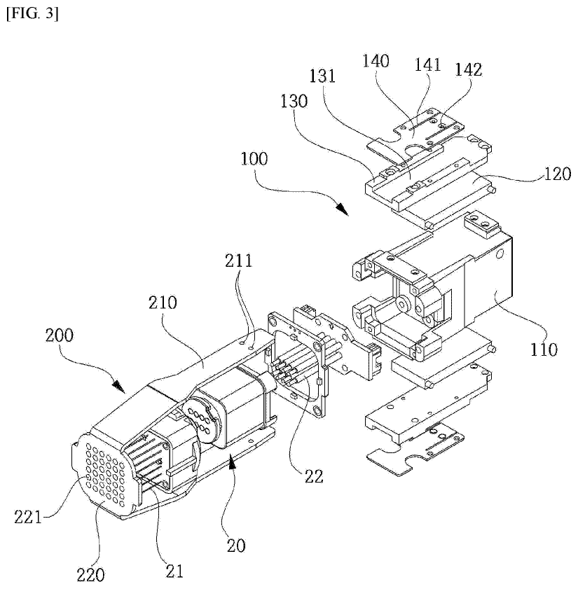

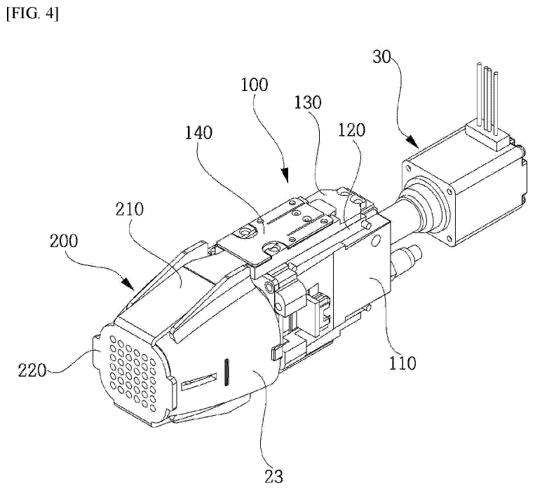

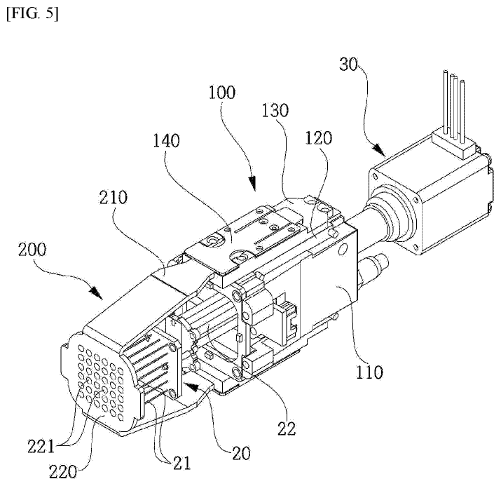

FIGS. 1 to 5 are diagrams illustrating a handpiece including a cooling device according to an exemplary embodiment of the present invention.

A handpiece 1 including a cooling device according to an exemplary embodiment of the present invention is a handpiece for a skin care device used for improving or treating the skin, and includes a case 10 provided to for an appearance of the handpiece as illustrated in FIGS. 1 to 5.

The case 10 is provided as a body having a shape that is easy for a user to grab by hand, and is mainly provided with a straight structure.

The appearance structure of the case 10 may be expressed in various shapes with respect to the external structure, and may be modified or transformed.

The case 10 includes a needle part 20 including needles 21, and a linear motor unit 30 for inducing the needle part 20 to perform forward and backward linear motion.

Further, the case 10 includes a cooling device for providing a cooling function to a treatment site during skin treatment using the skin penetration of the needle 21.

The above-described components are the components which are disposed inside and outside the case 10 to establish a mutual coupling relationship.

The cooling device includes a cooling head part 100 and a cooling plate part 200.

The cooling device is provided to perform a heat exchange function by using the supply and circulation of coolant, and then transfer the heat-exchanged cooling energy to the treatment site of the skin to cool the skin.

To this end, a coolant movement line 40 is connected to the rear end of the case 10, so that the coolant is circulated and supplied, and the cooling device is provided to be used as a medium for performing a heat exchange function through an inflow of the coolant to the cooling head part 100.

The coolant movement line 40 may be connected to a main body including a coolant supply container of a skin care device through a coolant cutoff and supply connector 50 or may be connected to a coolant supply container provided separately, and various attempts are possible.

The cooling head part 100 is mounted on the inner tip of the case 10 and is a component for generating cooling energy by performing a heat exchange function by using coolant as a medium.

The cooling head part 100 includes a heat exchange block 110, a Peltier element 120, a cooling plate socket 130, and a plate-shaped elastic body 140.

The heat exchange block 110 is positioned at a fore-end inside of the case 10 and is provided as a through-type body, and a coolant line for inflow and circulation of the coolant is formed inside the heat exchange block 110.

In this case, the coolant line may be formed in a hole structure that communicates with the inside of the through-type body, and a connection tube or the like may be used.

The heat exchange block 110 is a component having a characteristic of being cooled by heat exchange through the coolant line, and is preferably made of an aluminum material in order to increase heat transfer efficiency for cooling energy.

The Peltier elements 120 are disposed in close contact with each of the upper and lower surfaces of the heat exchange block 110, and are provided in a pair structure.

The Peltier element 120 is a thermoelectric element having a heating surface that emits warm energy on one surface and a cooling surface that emits cold energy on the other surface, and each of the Peltier elements 120 is arranged so that the heating surface is in close contact with the heat exchange block 110 and the cooling surface is in close contact with the cooling plate socket 130.

The cooling plate sockets 130 are arranged in a pair structure, and are fixedly coupled to the heat exchange block 110 through fastening of fixing bolts in a state of being stacked on the Peltier elements 120, respectively.

In this case, the fixing bolt is preferably provided with engineering plastic to prevent heat loss while securing strength.

In the cooling plate socket 130, a plate insertion groove 131 for enabling the rear end of the cooling plate part 200 to be inserted and placed while being in contact with each other is formed at the center in the longitudinal direction of the cooling plate socket 130.

The plate insertion groove 131 of the cooling plate socket 130 is formed so that the cooling plate part 200, in particular, the cooling energy transfer plate 210, is inserted and disposed by a slide movement.

The cooling plate socket 130 is preferably made of a copper material to minimize heat transfer loss with respect to the cooling energy transferred to the cooling plate part 200.

It is more preferable to use pure copper for the cooling plate socket 130 and may have a configuration in which the surface of the cooling plate socket 130 is chrome-plated to prevent oxidation due to condensation and binding of foreign substances.

On the heat exchange block 110 to which the cooling plate socket 130 is fastened, a silicon pad may be seated and placed on the fastening portion to block and prevent heat loss due to insulation.

The plate-shaped elastic bodies 140 are arranged in a pair structure, and are seated on upper surfaces of the cooling plate sockets 130, respectively, and are interposed between the case 10 and the cooling plate sockets 130.

The plate-shaped elastic body 140 has a plate body configured to be seated and disposed on the upper surface of the cooling plate socket 130, and a pair of slit holes 141 is formed at intervals on the plate body for an elastic structure.

The plate-shaped elastic body 140 is provided with a metal material, but is preferably provided with a stainless material to exhibit a function of preventing heat loss.

The rear end of the cooling plate part 200 is in contact with the cooling head part 100 to receive the heat-exchanged cooling energy from the cooling head part 100 and the fore-end of the cooling plate part 200 is located in front of the case 10 and is exposed, so that the cooling plate part 200 cools the treatment site through the contact with the skin during the treatment for the skin care.

To this end, the cooling plate part 200 includes the cooling energy transfer plate 210 and a skin contact cooling plate 220.

In the cooling energy transfer plate 210, rear ends are inserted into and contacted with the cooling plate sockets 130 of the cooling head 100, respectively, and a fore-end is exposed to the outside of the case 10.

The cooling energy transfer plates 210 have a one-pair structure in which the cooling energy transfer plates 210 are positioned at upper and lower sides and disposed at an interval.

The skin contact cooling plate 220 is coupled to the fore-ends of the pair of cooling energy transfer plates 210 exposed to the outside to function as a connecting member, and is in direct contact with the skin during the procedure to cool the treatment site, and the needle through holes 221 for the penetration and the arrangement of the needles 21 are formed at positions corresponding to the positions of the needles and in the number corresponding to the number of needles.

At least one detachable recess 211 is formed on the outer surface of the rear end of each of the cooling energy transfer plates 210, and detachable protrusions 142 are formed at the positions corresponding to the positions of the detachable recesses 211 and in the number corresponding to the number of detachable recesses 211 in each of the plate-shaped elastic bodies 140.

In this case, it is preferable that the detachable recess 211 and the detachable protrusion 142 are formed in a circular structure so as to easily provide and release fixing force by a mutual matching structure.

That is, the detachable recess 211 and the detachable protrusion 142 has the structure in which when the cooling energy transfer plate 210 of the cooling plate part 200 is inserted into the cooling plate socket 130 and disposed, the detachable protrusion 142 of the plate-shaped elastic body 140 is inserted to the detachable recess 211 of the cooling energy transfer plate 210 to provide fixing force, and when the cooling energy transfer plate 210 is pulled out from the cooling plate socket 130, elasticity of the elastic plate-shaped elastic body 140 is induced by the pushing action by pulling external pressure, so that the matched and fixed state of the detachable protrusion 142 and the detachable recess 211 is easily released.

The cooling plate part 200 is preferably made of an aluminum material in order to increase the heat transfer efficiency for cold energy and the cooling efficiency according to skin contact.

The needle part 20 is positioned at the fore-end or in front of the heat exchange block 110 of the cooling head part 100, and includes the needles 21 for penetrating into the skin during skin care procedures.

The needle part 20 includes a connection terminal 22 for allowing an RF current to flow in the needle 21.

The needle part 20 is connected for inducing the needle 21 to linearly move in front and rear directions through the linear motor unit 30.

In the needle part 20, at least one bracket and the like may be used to connect the needle 21 and the linear motor unit 30, and the needle part 20 includes a needle tip 23 located between the cooling plate part 200 and the heat exchange block 110 while being positioned and coupled to the fore-end of the case 10.

The needle tip 23 functions to cover and protect the needle 21, and the needle through-holes that enable the needles 21 to penetrate are formed at the positions corresponding to the needles 21 and with the number of needles 21.

In the linear motor unit 30, a driving unit 32 disposed to penetrate the cooling head part 100, particularly, the heat exchange block 110, is coupled to the needle 21, a motor unit 31 for providing power is located at the rear of the heat exchange block 110 of the cooling head part 100, and the linear motor unit 30 is provided so as to induce the needle part 20 to perform the linear motion by the forward and backward moving of the driving unit 32 by the operation of the motor 31.

The case 10 may be provided with a first switch for providing an operation signal for the forward and backward movement of the linear motor unit 30, a second switch for supplying an RF current to the needle 21 or blocking the RF current, a third switch for providing an operation signal for supplying the coolant to the heat exchange block 110 and circulating the coolant, and the providing of the operation signal for the forward and backward movement of the linear motor unit 30, the supply of the RF current to the needle 21 or the block of the RF current, and the supply of the coolant to the heat exchange block 110 and the circulation of the coolant may be sequentially controlled by programming with one switch operation.

A use state and an action of the handpiece 1 including the cooling device according to the present invention including the foregoing configuration will be described below.

When the linear motor unit 30 is operated after the skin contact cooling plate 220 is located while being in contact with a desired area for skin care including a skin treatment, the driving unit 32 moves forwardly by receiving power from the motor unit 31, and the needles 21 connected to the driving unit 32 also move forwardly at the same time to penetrate into the skin.

The RF current is applied to the needles 21 in the state where the needles 21 penetrate into the skin to induce a condition change of the tissue, and coolant is supplied to and circulated in the heat exchange block 110 of the cooling head part 100.

In this case, the heat exchange block 110 serves to generate cooling energy through the heat exchange by the coolant and improve cooling performance of the Peltier element 120, and the cooling energy generated in the Peltier element 120 that is a thermoelectric element is transferred to the cooling plate socket 130.

Therefore, the cooling energy is transferred to the cooling energy transfer plate 210 of the cooling plate unit 200 that is in contact with and fixed to the cooling plate socket 130, and the cooling energy is finally transferred to the skin contact cooling plate 220 to cool a treatment site of the skin.

Herein, the cooling head part 100 and the cooling plate part 200 are designed to have heat transfer mechanism in which the Peltier element 120, the cooling plate socket 130, and the plate-type elastic body 140 are sequentially disposed on two facing surfaces of the heat exchange block 110 and heat is transferred in the state where the cooling plate part 200 is in contact with the cooling plate socket 130, thereby preventing and minimizing heat loss for the cooling device and thus improving cooling efficiency.

That is, it is possible to relieve pain and exhibit a skin soothing effect through the cooling action at the treatment side of the skin, and prevent thermal damage or burns of the treatment site.

The exemplary embodiment described above is only to describe exemplary embodiment of the present invention and is not limited to the exemplary embodiment, and various modifications and variations are possible by those skilled in the art within the spirit and claims of the present invention, and it will be said that the modifications and variations fall within the scope of the technical rights of the present invention.

What is claimed is:

1. A handpiece including a cooling device for a skin care device used for improving or treating a skin, the handpiece comprising:
a case provided for an appearance of the handpiece;
a cooling head part mounted at a fore-end inside the case and configured to perform a heat exchange function and generate cooling energy;
a cooling plate part which has a rear end that is in contact with the cooling head part to receive the heat-exchanged cooling energy from the cooling head part and has a fore-end that is positioned in front of the case and is exposed to cool a treatment site through the contact with the skin during a procedure for skin care;
a needle part which is located at a fore-end of the cooling head part or located in front of the cooling head part, and includes needles for penetrating into the skin during the procedure for skin care; and
a linear motor unit which includes a driving unit disposed so as to penetrate the cooling head part is coupled to the needle, and a motor unit located at a rear of the cooling head part, and induces a forward and backward linear movement of the driving unit.

2. The handpiece of claim 1, wherein a coolant movement line is connected to a rear end of the case to circulate and supply the coolant, and the coolant is introduced to the cooling head part and is used as a medium for performing a heat exchange function.

3. The handpiece of claim 1, wherein the cooling head part includes:
a heat exchange block which is located at a fore-end inside the case, has a through-type body, and is formed with a coolant line for inflow and circulation of a coolant;
one pair of Peltier elements disposed in close contact with upper and lower surfaces of the heat exchange block, respectively;
one pair of cooling plate sockets which are disposed in the Peltier elements in a stack form, respectively, are fastened and fixed to the heat exchange block, and have plate insertion grooves to which a rear end of the cooling plate part is inserted and placed while being in contact with the cooling plate socket; and
one pair of plate-type elastic bodies seated on upper surfaces of the cooling plate sockets, respectively, and interposed between the case and the cooling plate socket.

4. The handpiece of claim 3, wherein the cooling plate part includes:
one pair of cooling energy transfer plates of which rear ends are inserted into the cooling plate sockets of the cooling head part to be in contact with the cooling plate sockets, respectively, and fore-ends are exposed to the outside of the case, and which are located at upper and lower sides and disposed at an interval; and
a skin contact cooling plate which is coupled to the pore-ends of the one pair of cooling energy transfer plate exposed to the outside to function as connection members, and is in direct contact with the skin during procedure to serve to cool a treatment site, and is formed with needle through-holes for penetration and arrangement of the needles.

5. The handpiece of claim 4, wherein at least one detachment groove is formed in an external surface of a rear end of each of the cooling energy transfer plates, detachment protrusions are formed in each of the plate-type elastic bodies at positions corresponding to positions of the detachment grooves and in the number corresponding to the number of detachment grooves, and
in the cooling plate part, when the cooling energy transfer plate is inserted into the cooling plate socket, the detachment protrusion of the plate-type elastic body is inserted into the detachment groove of the cooling energy transfer plate to provide fixing force, and when the cooling energy transfer plate is pulled out from the cooling plate socket, a matched and fixed state of the detachment protrusion and the detachment groove is easily released by inducing elasticity of the plate-type elastic body by a push operation by external pressure.

6. The handpiece of claim 3, wherein the plate-type elastic body includes a plate body seated on an upper surface of the cooling plate socket, and is formed with one pair of slit holes disposed at an interval on the plate body for an elastic structure.

7. The handpiece of claim 3, wherein the heat exchange block is made of an aluminum material,
each of the Peltier elements is disposed so that a heating surface is in close contact with the heat exchange block and a cooling surface is in close contact with the cooling plate socket, and
the cooling plate socket is made of a copper material so as to minimize thermal transfer loss for the cooling energy transferred to the cooling plate part.
